(19) United States
(12) Patent Application Publication
(10) Pub. No.: US 2011/0054579 A1
(43) Pub. Date: Mar. 3, 2011

(54) FLEXIBLE PENETRATING ELECTRODES FOR NEURONAL STIMULATION AND RECORDING AND METHOD OF MANUFACTURING SAME

(75) Inventors: G. Krishna Kumar, Troy, MI (US); Joseph M. Chalil, Shelby Twp., MI (US); Nishit A. Choksi, Troy, MI (US)

(73) Assignee: Advanced Microfab, LLC, Troy, MI (US)

(21) Appl. No.: 12/547,191

(22) Filed: Aug. 25, 2009

(51) Int. Cl. A61N 1/05; B44C 1/22

(52) U.S. Cl. 607/116; 216/13

(57) ABSTRACT

A flexible penetrating array for neuronal applications includes an insulating layer. A conductive layer is formed on the insulating layer. A flexible polymer substrate is formed on the conductive layer; the polymer substrate includes defined penetrating electrodes. A first metallization layer is formed on the polymer substrate. A second flexible polymer layer is formed on the first metallization layer. A second metallization layer is formed on the second flexible polymer layer. A third flexible polymer layer is formed on the second metallization layer. The third flexible polymer layer is patterned to expose the second metallization layer that is integrated with the out of plane conductive layer and first metallization layer. Also disclosed is a method of forming the array.

### FIELD OF THE INVENTION

The invention relates to penetrating electrodes and a method of producing flexible penetrating multi-electrode arrays for neuronal applications.

### BACKGROUND OF THE INVENTION

Micro scale electrodes are known in the art and may be capable of stimulating and recording neural tissue. However known electrodes are generally 2D or surface electrodes and are manufactured by having a substrate layer usually an insulating polymer and consisting of a plurality of conductive electrodes fabricated on top of this. After coating another insulating polymer on these conducting electrodes, the conducting surface is exposed. 2D surface electrodes are limited in their ability to access various target surfaces and provide only limited access to different surfaces. It would however be desirable to realize 3-D penetrating electrodes as they could provide large charge transfer capability due to low electrode-electrolyte impedance and access various portions of a desired target.

Prior art penetrating electrodes currently are mostly made of silicon. Some of the drawbacks of the above silicon based penetrating stimulation electrodes is that they are complex in micro-fabrication and also their integration with electronics may require another flexible cable to be bonded and electrically connected using cumbersome methods like soldering etc. Another major disadvantage is that silicon has not been proved biocompatible. Additionally, silicon may trigger undesirable bodily reactions such the persistence of macrophages surrounding chronically or long term implanted neuroprosthetic devices and deleterious effects on adjacent nerve cell bodies and their processes.

Another problem with prior art penetrating movement of the penetrating rigid electrode array inside the soft tissue causing significant damage. There is therefore a need in the art for an improved array that is flexible to conform to various shaped targets and biocompatible. There is also a need in the art for an array that is easily mated with a microelectronic device.

### SUMMARY OF THE INVENTION

In one aspect there is disclosed a method of forming a flexible penetrating array for neuronal applications including the steps of: providing a substrate; forming at least one opening in the substrate; applying at least one insulating layer overlying the opening and the substrate; applying at least one patterned conductive layer overlying the insulating layer; applying a first polymer layer overlying the conductive layer filling the opening and overlying the substrate; patterning at least one via on the first polymer layer accessing the conductive layer; applying at least one patterned metallization layer overlying the first polymer layer and in electrical contact with the conductive layer; applying at least one secondary polymer material overlying the first polymer layer sandwiching the metallization layer; patterning the substrate forming a second opening and etching the insulating layer; applying at least one secondary metallization layer overlying the conductive layer; and applying a third polymer layer overlying the entire array with at least one via opening to access the secondary metallization layer.

In another aspect there is disclosed a flexible penetrating array for neuronal applications that includes an insulating layer. A conductive layer is formed on the insulating layer. A flexible polymer substrate is formed on the conductive layer; the polymer substrate includes defined penetrating electrodes. A first metallization layer is formed on the polymer substrate. A second flexible polymer layer is formed on the first metallization layer. A second metallization layer is formed on the second flexible polymer layer. A third flexible polymer layer is formed on the second metallization layer. The third flexible polymer layer is patterned to expose the second metallization layer that is integrated with the out of plane conductive layer and first metallization layer.

### BRIEF DESCRIPTION OF THE DRAWINGS

FIG. 1a-1b is a cross-sectional view showing the process flow for forming a flexible penetrating electrode array by etching trenches in the substrate and applying a conductor and insulator conforming to the trench;

FIG. 2a-2b is a cross-sectional view showing the process flow for forming a flexible penetrating electrode array by filling the trench with parylene and opening up the via to the underlying conductor to couple the metallization layer;.

FIG. 3a-3b is a cross-sectional view showing the process flow for forming a flexible penetrating electrode array by applying another parylene layer overlying the metallization conductive layer, opening the backside of the substrate to access the conductive layer, penetrating portion and subsequently depositing another metallization layer;

FIG. 4a-4b is a cross-sectional view showing the process flow for forming a flexible penetrating electrode array releasing the device from the carrier substrate and subsequently applying another insulating parylene layer and opening the vias;

FIG. 5a-5b is a cross-sectional view showing the process flow for forming a flexible penetrating electrode array showing the bonding of the released device to a backing substrate layer;

FIG. 6 is a 3D view of the flexible penetrating array showing the matrix of penetrating electrodes.

### DETAILED DESCRIPTION OF THE PREFERRED EMBODIMENTS

Referring to the various figures, the present disclosure relates to flexible penetrating, multi-electrode arrays 10 for neural stimulation and recording and the method of manufacture for the same. The flexible nature of the arrays 10 includes the use of a polymer that is flexible and bio-compatible. In one aspect the polymer may include parylene. The term penetrating may also describe micro-needles and they are used interchangeably in this disclosure. Parylene is a United States Pharmacopoeia (USP) Class VI biocompatible material that has good barrier properties against, strong acids, inorganic and organic substances and water vapor. Parylene is a bio-stable and biocompatible material approved by the FDA for various applications. Manufacturing is also cost effective with various deposition techniques including CVD, or Chemical Vapor Deposition which takes place at low pressure and at room temperature. Parylene may also be etched in an Oxygen plasma environment using RIE (Reactive Ion etching). Various forms of parylene include: N, C, D, F, and HT. In one aspect, parylene C may be utilized.

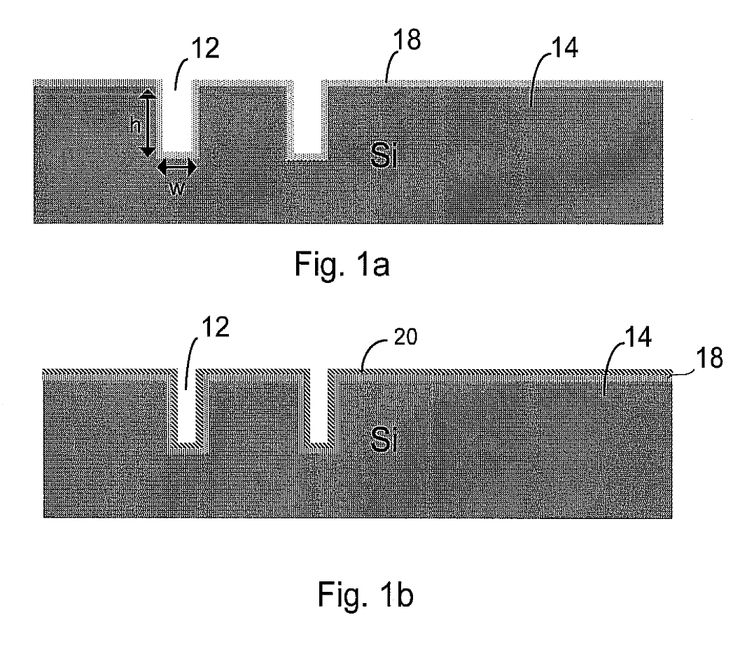

The microfabrication for the penetrating flexible electrode array 10 starts by etching a trench 12 in a silicon substrate 14 as shown in FIG. 1a. The trench 12 may be etched by DRIE or any other wet chemical etching to give the shape of the penetrating electrode 16. The dimensions of the trench including the height “h” and width “w” define the dimensions and the shape of the penetrating electrode 16. The height “h” may range from tens of microns to 1.5 mm and the width “w” may range from 5 microns to tens of microns. A person of ordinary skill in this art will be able to easily make further alterations and modifications after reading the present invention. It can easily be inferred that any particular embodiment illustrated with diagrams and explained cannot be considered limiting. Modifications to the current embodiment may include etching the trench 12 through the entire thickness of the substrate 14 or forming the trench 12 in such a way to have slanted walls or tips.

Again referring to FIG. 1a, in this specific embodiment, next an insulating layer 18 such as silicon dioxide may be deposited to conform to the trench 12 and also to the silicon substrate 14. In one aspect the silicon dioxide may be deposited by LPCVD. The insulating layer 18 may be utilized as a sacrificial layer to release the completed device from the silicon substrate 14. The insulating layer 18 may have a thickness of from one micron to several microns.

Next, a conductive layer of polysilicon 20 may be deposited overlying the insulating layer 18 as shown in FIG. 1b. In one aspect the polysilicon may be deposited by LPCVD. The polysilicon layer 20 formed on the silicon dioxide layer 18 may be patterned to have a defined pattern. The polysilicon layer 20 may be doped with boron or phosphorous to make it conductive and aid in defining an electrical layer on the penetrating electrode array 10 that will be used for neuronal stimulation or recording. A person of ordinary skill in this art will be able to easily make further alterations and modifications after reading the present invention. It can easily be inferred that any particular embodiment illustrated with diagrams and explained cannot be considered limiting. For example the sequence of forming the sacrificial and conductive layers can be added or subtracted or the sequence changed.

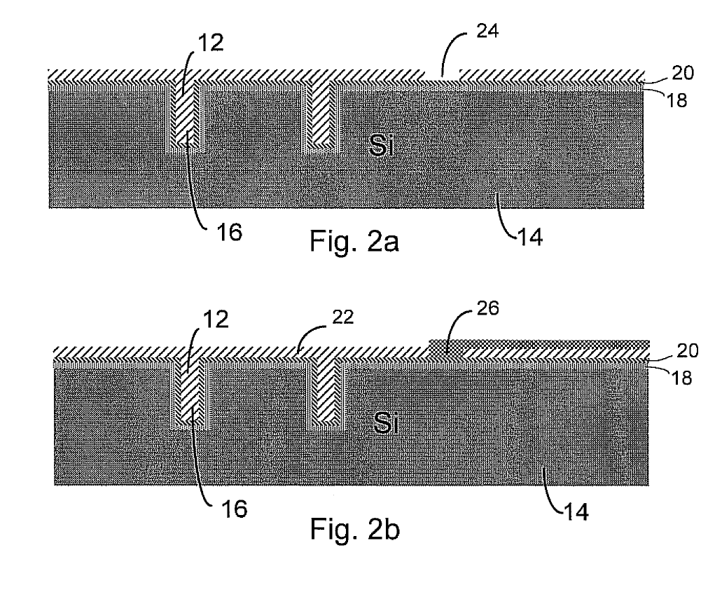

Next as shown in FIG. 2a, a polymer layer 22 may be applied over the polysilicon layer 20. In one aspect the polymer layer 22 may be a layer of Parylene C that is formed to fill in the trench 12 and act as the mechanical penetrating electrode 16. Since the deposition of parylene is conformal the polymer or parylene C layer is formed on the silicon substrate 14 as well and this will define the substrate for the final electrode array 10. The deposition process forms a 3D profile to the electrode array 10.

Next, the parylene C layer 22 formed overlying the conductive layer or polysilicon layer 20 may be patterned and etched in an oxygen plasma environment using Reactive Ion Etching (RIE) to define a via 24. The via 24 allows access to the conductive layer 20.

A metallization layer 26 may then be formed using a defined pattern on the parylene layer 22 that also connects to the polysilicon conductive layer 22 through the via 24. The metallization layer 26 may be formed of materials including: metals such as, aluminum, copper, titanium, chrome, gold, silver, iridium or their combination that can be evaporated, sputtered or electroplated. The metallization layer 26 provides electrical contact to the conductive layer 20 on the penetrating electrode 16 and provides access for the array 10 to be interfaced with electronics. A person of ordinary skill in this art will be able to easily make further alterations and modifications after reading the present invention. It can easily be inferred that any particular embodiment illustrated with diagrams and explained cannot be considered limiting. For example the trench 12 may be filled by other materials including polymers such as polyimide and then be coated with parylene, the sequence of steps may be changed or modified and more than one metallization layer can be formed.

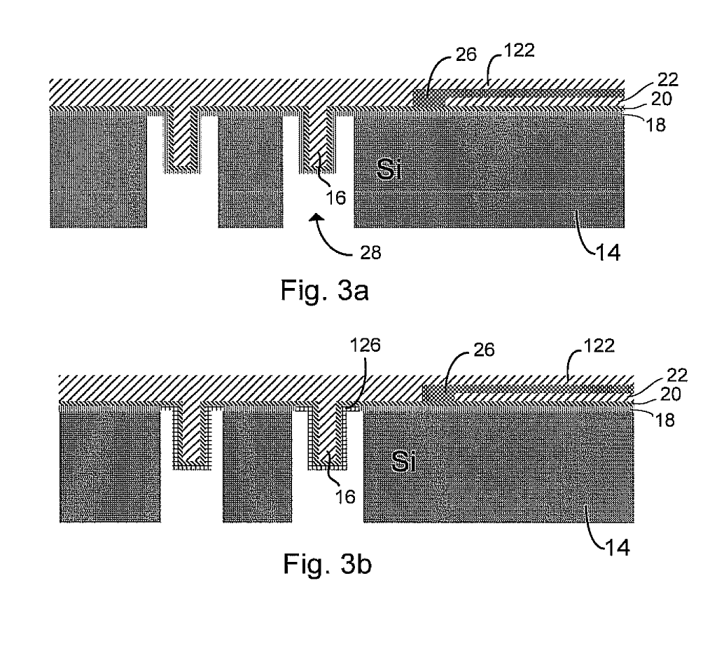

Referring back to FIG. 3a, another polymer layer 122 that may be formed of parylene C is then formed sandwiching the metallization layer 26. The thickness of the polymer layer 122 may range from a couple of microns to several tens of microns. The backside of the silicon substrate 14 may then be patterned in a defined manner to access the penetrating electrode array by forming an opening 28 using DRIE. The formation of this opening 28 may aid in releasing the penetration part of the electrode 16 from the silicon substrate 14.

Next, the insulating layer 18 may be etched in a wet etchant such as Buffered Hydrofluoric acid as shown in FIG. 3b. Another metallization layer 126 may then be deposited from the backside and be patterned onto the penetrating portion of the electrode 16. The metallization layer 126 may be formed of metals such as, aluminum, copper, titanium, chrome, gold, silver, iridium or their combination that can be evaporated, sputtered or electroplated. A person of ordinary skill in this art will be able to easily make further alterations and modifications after reading the present invention. It can easily be inferred that any particular embodiment illustrated with diagrams and explained cannot be considered limiting. For example the sequence of forming the metallization layers onto the penetrating portion of the electrode 16 can be added or subtracted or the sequence changed.

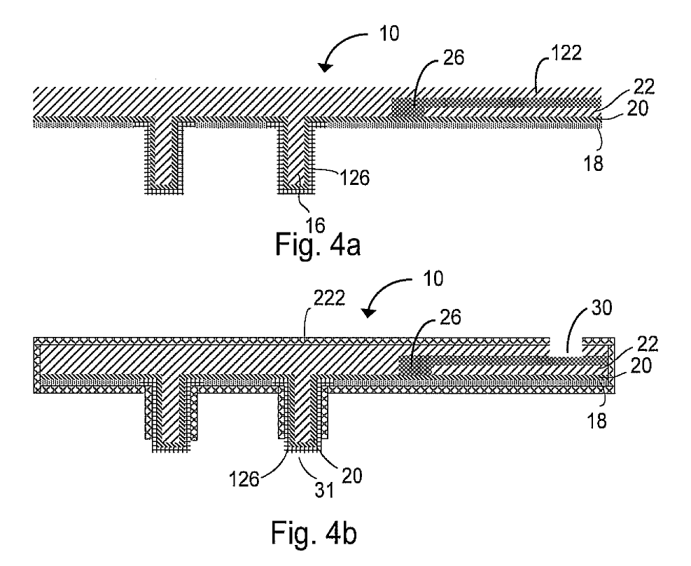

Referring to FIG. 4a, the array 10 may be released from the silicon substrate 14. A further polymer layer 222 such as parylene may be formed overlying the entire array 10. The polymer layer 222 may then be patterned to form a via 30 to connect to outside electronics and also form an opening 31 to expose the tip 40 of the penetrating electrode 16 that includes the metallization layer 126.

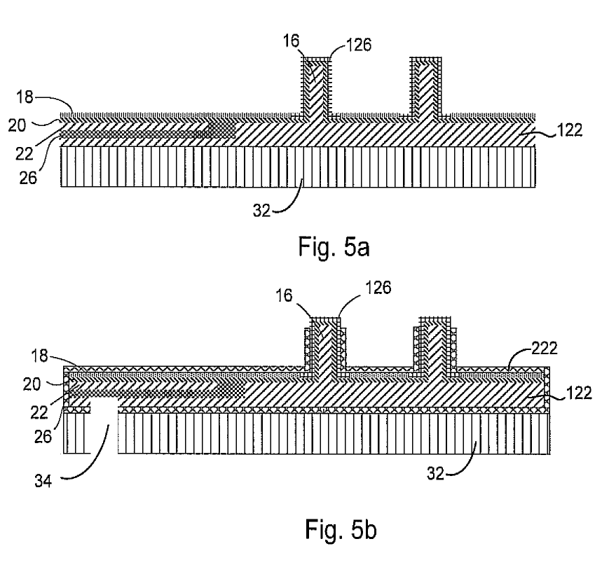

In an alternative embodiment shown in FIG. 5a, after releasing the array 10 from the silicon substrate 14, the flexible penetrating electrode array 10 may be mated to a carrier substrate 32. The carrier substrate 32 may be formed of a flexible material such as, silicone or rigid materials such as metal alloys or semi conductive substrates. After mating the released array 10 to the carrier substrate 32 a new via 34 may be formed to access the metallization layer 126 as shown in FIG. 5b.

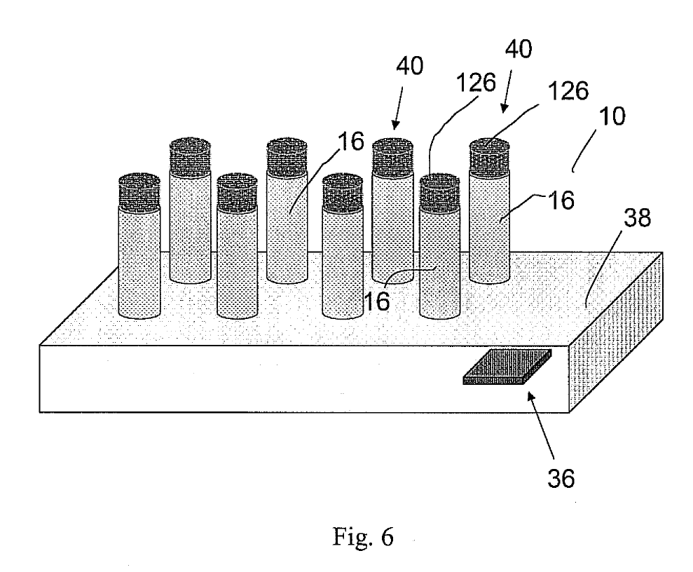

Referring to FIG. 6, there is shown a completed array 10 including the penetrating electrodes 16. The array is shown mated to an electronic device 36 that may record signals or provide stimulation. As can be seen in the figure, the array 10 includes penetrating electrodes 16 that are formed of a flexible polymer allowing movement of the electrodes 16 to conform to various shapes. Additionally, the base 38 of the array 10 is also formed of a flexible material. The tips 40 of the electrodes 16 include a metallization layer 126 that may conduct signals or electro pulses to or from a substance that interfaces with the array 10.

The array 10 overcomes problems in the prior art through the utilization of a flexible polymer material that is biocompatible. The penetrating electrode 16 is surrounded by a thin conductive layer of LPCVD polysilicon 20. The flexible polymer forms the base layer of the array 10 providing flexibility to the entire device. The flexible polymer provides both the penetrating portion and the base because of the conformal coating of the deposition process. Since the penetrating part of the array is made of a flexible polymer such as parylene it eliminates the problem of the prior art with the ability to move along with the soft tissue leading to minimal or no tissue damage. The use of parylene provides flexibility and also mechanical strength with high tensile and yield strength.

Another difficulty with prior art 3D arrays or penetrating electrodes is the electrical interface. This invention further discloses a method to integrate out of plane or 3D conductors that are present on the penetrating electrode to the conductor present on the planar substrate in a very simple way. As described above, LPCVD polysilicon is first deposited to coat conforming to the shape of the penetrating array which is later insulated with parylene with the tip being exposed for neural interface. Parylene deposition which is conformal is then deposited to provide the mechanical rigidity to polysilicon and also the penetrating part of the electrode array. Parylene would also serve as the substrate or base layer. To access the LPCVD conductor present on the penetrating portion, a via is etched on the parylene and metal is deposited and patterned to make electrical contact. Parylene has also been proven to be compatible with Integrated circuits and this will simplify the integration of the array with various microelectronic devices. The flexible properties of the polymer layer such as parylene allows the penetrating array to easily conform to any non planar surface such as the cylindrical or spherical surface of nerves.

1. A method of forming a flexible penetrating array for neuronal applications comprising the steps of:
providing a substrate;
forming at least one opening in the substrate;
applying at least one insulating layer overlying the opening and the substrate;
applying at least one patterned conductive layer overlying the insulating layer;
applying a first polymer layer overlying the conductive layer filling the opening and overlying the substrate;
patterning at least one via on the first polymer layer accessing the conductive layer;
applying at least one patterned metallization layer overlying the first polymer layer and in electrical contact with the conductive layer;
applying at least one secondary polymer material overlying the first polymer layer sandwiching the metallization layer;
patterning the substrate forming a second opening and etching the insulating layer;
applying at least one secondary metallization layer overlying the conductive layer;
applying a third polymer layer overlying the entire array with at least one via opening to access the secondary metallization layer.

2. The method of claim 1 including the step of applying at least one backing substrate layer.

3. The method of claim 1 wherein the conductive layer is non planar.

4. The method of claim 1 wherein the metallization layer is planar.

5. The method of claim 1 wherein the opening is formed using DRIE or another wet chemical etching including TMAH and KOH.

6. The method of claim 1 wherein the insulating layer includes a LPCVD oxide or nitride.

7. The method of claim 1 wherein the conducting layer is selected from: LPCVD polysilicon, metals including titanium, chrome, gold, platinum, and iridium.

8. The method of claim 7 wherein the conductive layer is patterned by photolithographically defining selective areas and etching the conductive layer in exposed areas using wet or dry etching.

9. The method of claim 1, wherein the first polymer layer includes parylene C.

10. The method of claim 1, wherein the via is formed by etching including Reactive Ion etching and laser ablation.

11. The method of claim 1, wherein the metallization layer is formed of metals selected from: titanium, chrome, gold, platinum, iridium or their combination that can be evaporated, sputtered or electroplated.

12. The method of claim 1 wherein the metallization layer is patterned by photolithographically defining selective areas and removing the metallization layer in exposed areas.

13. The method of claim 1, wherein the said second polymer layer is selected from: parylene C, polyimide, and silicone.

14. The method of claim 1, wherein the secondary metallization layer is selected from: metals including, aluminum, copper, titanium, chrome, gold, silver, iridium or their combination that can be evaporated, sputtered or electroplated.

15. The method of claim 1, wherein the third polymer layer includes parylene C.

16. The method of claim 2, wherein the backing substrate layer is selected from conductive including metal alloys, non-conductive, and insulating materials.

17. A flexible penetrating array for neuronal applications comprising:
an insulating layer;
a conductive layer formed on the insulating layer;
a flexible polymer substrate formed on the conductive layer, the polymer substrate including defined penetrating electrodes;
a first metallization layer formed on the polymer substrate;
a second flexible polymer layer formed on the first metallization layer;
a second metallization layer formed on the second polymer layer;
a third flexible polymer layer formed on the second metallization layer
wherein the third polymer layer is patterned to expose the second metallization layer that is integrated with the out of plane conductive layer and first metallization layer.

18. The flexible penetrating array of claim 17 including a via formed in the second and third polymer layers exposing the first metallization layer.

19. The flexible penetrating array of claim 18 including a micro electronic device integrated at the via to the first metallization layer.

20. The flexible penetrating array of claim 17 wherein the first, second and third polymer layers are formed of parylene.
